(19) United States
(12) Patent Application Publication
(10) Pub. No.: US 2022/0246246 A1
(43) Pub. Date: Aug. 4, 2022

(54) PROCESS CONFIGURATION GENERATION AND SIMULATION

(71) Applicant: International Business Machines Corporation, Armonk, NY (US)

(72) Inventors: Pedro Arrechea, San Jose, CA (US); Dmitry Zubarev, San Jose, CA (US); Nathaniel H. Park, San Jose, CA (US); James L. Hedrick, Pleasanton, CA (US); Tim Erdmann, San Jose, CA (US)

(21) Appl. No.: 17/165,094

(22) Filed: Feb. 2, 2021

(51) Int. Cl. G16C 20/10; G16C 20/70; G06F 30/27; G06N 20/00

(52) U.S. Cl. CPC G16C 20/10; G16C 20/70; G06F 2111/10; G06N 20/00; G06F 30/27

(57) ABSTRACT

Commands and constraints of reconfigurable instrumentation that execute unit operations of processes can be defined. A simulation engine configured to execute commands of the reconfigurable instrumentation can be defined such that processes can be simulated based on pre-defined mathematical models. A process historian configured to monitor and record results of the simulation can be defined. A first process can be simulated based on a first process configuration. The first process configuration can be mapped onto real-world reconfigurable instrumentation within the first process configuration.

### BACKGROUND

The present disclosure relates generally to the field of control systems, and in particular, to generating and simulating process configurations.

In the chemical engineering field, processes are used to obtain desired products from starting materials (e.g., chemical compounds). A process may include many steps, each of which can include one or more unit operations. Unit operations can involve a transformation (e.g., physical or chemical) or measurement of a particular species during a particular stage of the process. Example unit operations include homogenization, phase separation, chemical reaction, chromatography, evaporation, pressure change, temperature change, distillation, extraction, and others.

To reach a desired goal within a given process, many variables within the process can be altered. For example, variables such as the number of unit operations, type of unit operations, timing of unit operations, order of unit operations, chemical species characteristics (e.g., concentration), mass flow, pressure, temperature, and other variables can be adjusted within a given process to improve the yield/acquisition/quality of a given product. Identifying the correct set-up for a process is typically completed through traditional methods such as trial and error and/or mathematical calculations. Engineers are typically required to start with constraints on the type and amount of equipment available to them. As a result, many preferred set-ups to reach a desired goal may not be successfully identified due to the methods used to identify the process as well as the initial constraints on the system.

### SUMMARY

Embodiments of the present disclosure include a method for process generation and simulation. Commands and constraints of reconfigurable instrumentation that execute unit operations of processes can be defined. A simulation engine configured to execute commands of the reconfigurable instrumentation can be defined such that processes can be simulated based on pre-defined mathematical models. A process historian configured to monitor and record the results of the simulation can be defined. A first process can be simulated based on a first process configuration. The first process configuration can be mapped onto real-world reconfigurable instrumentation within the first process configuration.

The above-referenced method allows users to test (e.g., via computer simulation) process configurations prior to set-up in a real-world environment. This allows users to save implementation cost on set-ups that may be required to be modified in the future. Further, this allows users to find a preferred set-up which may meet their product/process goals.

The method can further include revising the first process by altering the first process configuration and mapping the revised process configuration onto the real-world reconfigurable instrumentation within the revised process configuration.

This allows users to identify revised versions of processes over time without requiring users to actually test the process in the real-world. This saves cost and improves process performance (e.g., product quality and/or quantity).

Embodiments of the present disclosure further include a system including one or more processors and one or more computer-readable storage media storing program instructions which, when executed by the one or more processors, are configured to cause the one or more processors to perform a method for process generation and simulation. The method can include defining commands and constraints of reconfigurable instrumentation that execute unit operations of processes. The method can further include defining a simulation engine configured to execute commands of the reconfigurable instrumentation such that processes can be simulated based on pre-defined mathematical models. The method can further include defining a process historian configured to monitor and record the results of the simulation. The method can further include simulating a first process based on a first process configuration. The method can further include mapping the first process configuration onto real-world reconfigurable instrumentation within the first process configuration.

The above-referenced system allows users to test (e.g., via computer simulation) process configurations prior to set-up in a real-world environment. This allows users to save implementation cost on set-ups that may be required to be modified in the future. Further, this allows users to find a preferred set-up which may meet their product/process goals.

The method performed by the one or more processors can further include revising the first process by altering the first process configuration and mapping the revised process configuration onto the real-world reconfigurable instrumentation within the revised process configuration.

This allows users to identify revised versions of processes over time without requiring users to actually test the process in the real-world. This saves cost and improves process performance (e.g., product quality and/or quantity).

Embodiments of the present disclosure further include a computer program product comprising one or more computer readable storage media, and program instructions collectively stored on the one or more computer readable storage media, the program instructions comprising instructions configured to cause one or more processors to perform a method for process generation and simulation. The method can include defining commands and constraints of reconfigurable instrumentation that execute unit operations of processes. The method can further include defining a simulation engine configured to execute commands of the reconfigurable instrumentation such that processes can be simulated based on pre-defined mathematical models. The method can further include defining a process historian configured to monitor and record the results of the simulation. The method can further include simulating a first process based on a first process configuration. The method can further include mapping the first process configuration onto real-world reconfigurable instrumentation within the first process configuration.

The above-referenced computer program product allows users to test (e.g., via computer simulation) process configurations prior to set-up in a real-world environment. This allows users to save implementation cost on set-ups that may be required to be modified in the future. Further, this allows users to find a preferred set-up which may meet their product/process goals.

The method performed by the one or more processors can further include revising the first process by altering the first process configuration and mapping the revised process configuration onto the real-world reconfigurable instrumentation within the revised process configuration.

This allows users to identify revised versions of processes over time without requiring users to actually test the process in the real-world. This saves cost and improves process performance (e.g., product quality and/or quantity).

The above summary is not intended to describe each illustrated embodiment or every implementation of the present disclosure.

### BRIEF DESCRIPTION OF THE DRAWINGS

The drawings included in the present disclosure are incorporated into, and form part of, the specification. They illustrate embodiments of the present disclosure and, along with the description, serve to explain the principles of the disclosure. The drawings are only illustrative of typical embodiments and do not limit the disclosure.

FIG. 1 is a block diagram illustrating an example computing environment in which illustrative embodiments of the present disclosure can be implemented.

FIG. 2 is a block diagram illustrating a distributed control system, in accordance with embodiments of the present disclosure.

FIG. 3 is a flow diagram illustrating an example method for configuring and utilizing a distributed control system, in accordance with embodiments of the present disclosure.

FIG. 4 is a flow diagram illustrating an example method for improving an initial process configuration using reinforcement learning, in accordance with embodiments of the present disclosure.

FIG. 5 is a flow diagram illustrating an example method for improving an initial process configuration using supervised learning, in accordance with embodiments of the present disclosure.

FIG. 6 is a diagram illustrating a cloud computing environment, in accordance with embodiments of the present disclosure.

FIG. 7 is a block diagram illustrating abstraction model layers, in accordance with embodiments of the present disclosure.

FIG. 8 is a high-level block diagram illustrating an example computer system that may be used in implementing one or more of the methods, tools, and modules, and any related functions, described herein, in accordance with embodiments of the present disclosure.

While the embodiments described herein are amenable to various modifications and alternative forms, specifics thereof have been shown by way of example in the drawings and will be described in detail. It should be understood, however, that the particular embodiments described are not to be taken in a limiting sense. On the contrary, the intention is to cover all modifications, equivalents, and alternatives falling within the spirit and scope of the disclosure.

### DETAILED DESCRIPTION

Aspects of the present disclosure relate generally to the field of control systems, and in particular, to simulating and generating process configurations. While the present disclosure is not necessarily limited to such applications, various aspects of the disclosure can be appreciated through a discussion of various examples using this context.

In the chemical engineering field, processes are used to obtain desired products from starting materials (e.g., chemicals). A process may include many steps, each of which can include one or more unit operations. Unit operations can involve a transformation (e.g., physical or chemical) or measurement of a particular species during a particular stage of the process. Example unit operations include homogenization, phase separation, chemical reaction, chromatography, evaporation, pressure change, temperature change, distillation, extraction, and others. Various instrumentation can be used to carry out these unit operations.

To reach a desired goal within a given process, many variables within the process can be altered. For example, variables such as the number of unit operations, type of unit operations, timing of unit operations, order of unit operations, chemical species characteristics (e.g., concentration), mass flow, pressure, temperature, and other variables can be adjusted within a given process to improve the yield/acquisition/quality of a given product. Identifying the correct set-up for a process is typically completed through traditional methods such as trial and error and/or mathematical calculations. Engineers are typically required to start with constraints on the type and amount of equipment available to them. As a result, many preferred set-ups to reach a desired goal may not be successfully identified due to the methods used to identify the process as well as the initial constraints on the system.

Aspects of the present disclosure relate to process generation and simulation. Commands and constraints of reconfigurable instrumentation that execute unit operations of processes can be defined. A simulation engine configured to execute commands of the reconfigurable instrumentation can be defined such that processes can be simulated based on pre-defined mathematical models. A process historian configured to monitor and record the results of the simulation can be defined. A first process can be simulated based on a first process configuration. The first process configuration can be mapped onto real-world reconfigurable instrumentation within the first process configuration.

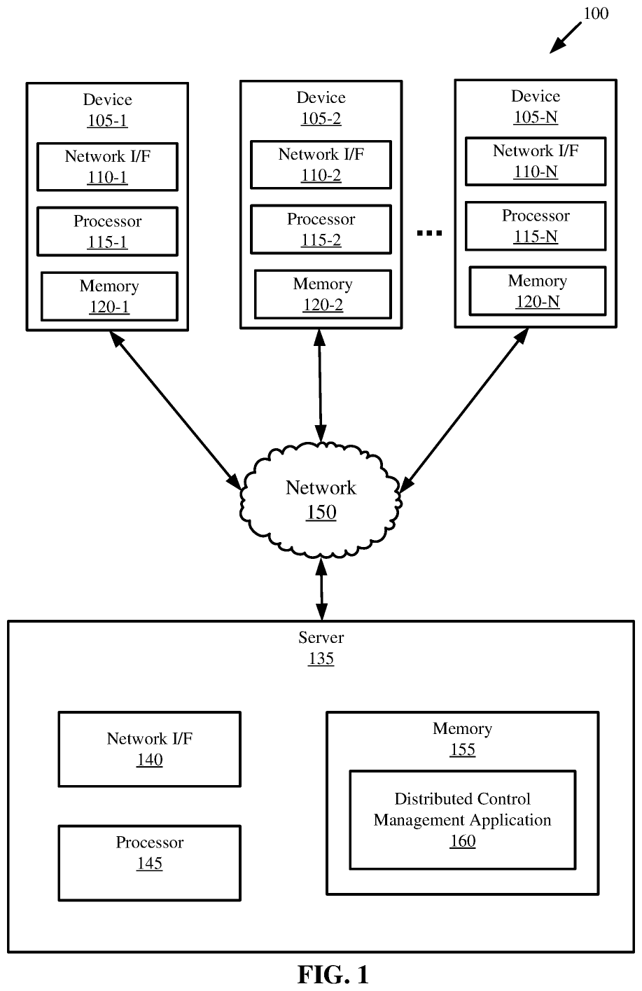

Turning now to the figures, FIG. 1 is a block diagram illustrating an example computing environment 100 in which illustrative embodiments of the present disclosure can be implemented. Computing environment 100 includes a plurality of devices 105-1, 105-2 . . . 105-N (collectively devices 105), at least one server 135, and a network 150.

Consistent with various embodiments, the server 135 and the devices 105 are computer systems. The devices 105 and the server 135 include one or more processors 115-1, 115-2 . . . 115-N (collectively processors 115) and 145 and one or more memories 120-1, 120-2 . . . 120-N (collectively memories 120) and 155, respectively. The devices 105 and the server 135 can be configured to communicate with each other through internal or external network interfaces 110-1, 110-2 . . . 110-N (collectively network interfaces 110) and 140. The network interfaces 110 and 140 are, in some embodiments, modems or network interface cards. The devices 105 and/or the server 135 can be equipped with a display or monitor. Additionally, the devices 105 and/or the server 135 can include optional input devices (e.g., a keyboard, mouse, scanner, video camera, or other input device), and/or any commercially available or custom software (e.g., browser software, communications software, server software, natural language processing software, search engine and/or web crawling software, image processing software, etc.). The devices 105 and/or the server 135 can be servers, desktops, laptops, or hand-held devices.

The devices 105 and the server 135 can be distant from each other and communicate over a network 150. In some embodiments, the server 135 can be a central hub from which devices 105 can establish a communication connection, such as in a client-server networking model. Alternatively, the server 135 and devices 105 can be configured in any other suitable networking relationship (e.g., in a peer-to-peer (P2P) configuration or using any other network topology).

In some embodiments, the network 150 can be implemented using any number of suitable communications media. For example, the network 150 can be a wide area network (WAN), a local area network (LAN), an internet, or an intranet. In certain embodiments, the devices 105 and the server 135 can be local to each other and communicate via any appropriate local communication medium. For example, the devices 105 and the server 135 can communicate using a local area network (LAN), one or more hardwired connections, a wireless link or router, or an intranet. In some embodiments, the devices 105 and the server 135 can be communicatively coupled using a combination of one or more networks and/or one or more local connections. For example, the first device 105-1 can be hardwired to the server 135 (e.g., connected with an Ethernet cable) while the second device 105-2 can communicate with the server 135 using the network 150 (e.g., over the Internet).

In some embodiments, the network 150 is implemented within a cloud computing environment or using one or more cloud computing services. Consistent with various embodiments, a cloud computing environment can include a network-based, distributed data processing system that provides one or more cloud computing services. Further, a cloud computing environment can include many computers (e.g., hundreds or thousands of computers or more) disposed within one or more data centers and configured to share resources over the network 150.

In embodiments, the devices 105 can be instrumentation used to carry out steps (e.g., unit operations) within a process. For example, devices 105 can include instrumentation such as reactors (e.g., batch, continuous, plug flow), fermenters, filters, centrifuges, crystallizers, condensers, adsorbers, storage tanks, homogenizers (e.g., high pressure, bead mill, nano-mill), dryers (e.g., freeze dryers), evaporators, pumps, compressors, blowers, heaters, coolers, separators, precipitators, and other instrumentation. The instrumentation or actuators (e.g., valves) associated therewith can be configured to receive commands from the server 135 to adjust one or more settings thereof (e.g., to alter flow rate, residence time, power, speed, etc.).

The server 135 includes a distributed control management application 160. The distributed control management application 160 can be a software-based environment in which unit operations for a given process can be defined, mass flow of particular species (e.g., chemical compounds) into the unit operations can be defined, the overall process including the unit operations can be simulated, data related to the simulations can be stored and analyzed for the purpose of improving the process configuration (e.g., mass flow, number of unit operations, type of unit operations, order of unit operations, etc.), and, upon selection of a particular process configuration, the process configuration can be mapped onto real-world objects (e.g., instrumentation configured to carry out the process can be remotely controlled such that the process can be executed).

The distributed control management application 160 can be configured to receive scripts (e.g., computer instructions defining executable commands) available to unit operations that can be used within processes. Unit operations can include operations such as homogenization, phase separation, chemical reaction, chromatography, evaporation, pressure change, separation, temperature change, and others. The scripts can depend on the functionality of the instrumentation defined within the distributed control management application 160. For example, defined scripts for a first unit operation (e.g., a continuous stirred-tank reactor (CSTR)) can include scripts to control actuators associated therewith, such as valve controls (e.g., “Valve Open” or “Valve Close”) and/or mix controls (e.g., “Set Mix Speed,” “Set Mix Type,” or “Set Mix Time”). The scripts allow users to control the unit operations throughout the process. The reagents (e.g., chemical compounds, mixtures, etc.) available to the distributed control management application to be fed into unit operations can also be defined. In embodiments, the cost of resources (e.g., cost of instrumentation and chemicals), physical attributes (e.g., dimensions), and/or environmental impact of resources within the distributed control management application 160 can also be defined. This can be used for the purpose of considering cost, physical space occupied, and/or environmental impact during process configuration design.

Thereafter, a user can design a process (e.g., a chemical plant) by defining the unit operations, mass flow, input reagents (e.g., type of compounds, concentration, etc.), residence times, temperatures, pressures, etc. associated with the process within a graphical user interface (GUI) of the distributed control management application 160. The process can then be simulated (e.g., based on mathematical models) such that the chemical compositions throughout the process can be calculated. The experimental data associated with the simulation can then be stored for analysis. Such data can include data such as residence times, chemical compositions (e.g., concentration, volume, mass, etc.), flow rates, analytical data (e.g., spectra captured by analytical instruments), commands executed, and/or other data throughout various time points within the process.

A user can then reconfigure variables defined within the process based on an analysis of the simulation. For example, the user can manually change one or more flow rates, chemical compositions, timings, unit operation settings, etc., defined within the process configuration. In embodiments, the distributed control management application 160 can include embedded artificial intelligence (AI) capabilities such that the process configuration can be improved based on the data received from the simulation. For example, in some embodiments, reinforcement learning capabilities can be integrated within the distributed control management application 160 such that a desired goal (e.g., based on yield, quality, cost, environmental impact, physical space occupied, etc.) can be attained based on incremental changes and observed reward associated with the state changes. As another example, in some embodiments, supervised learning (e.g., active learning) can be used such that the user can label desired outcomes throughout processes and receive AI-based modifications based on the supervised learning data.

Thereafter, upon selection of a desired process configuration, the distributed control management system 160 can be configured to map the process onto reconfigurable instrumentation (e.g., devices 105) such that the process can be executed in the real-world. That is, the unit operation settings, flow rates, residence times, etc., of instrumentation in the real-world can be adjusted (e.g., over network 150) based on the selected configuration. In embodiments, users may be required to manually adjust one or more variables within the process (e.g., a user may be required to formulate a particular chemical compound or mixture for input into the process).

It is noted that FIG. 1 is intended to depict the representative major components of an example computing environment 100. In some embodiments, however, individual components can have greater or lesser complexity than as represented in FIG. 1, components other than or in addition to those shown in FIG. 1 can be present, and the number, type, and configuration of such components can vary.

While FIG. 1 illustrates a computing environment 100 with a single server 135, suitable computing environments for implementing embodiments of this disclosure can include any number of servers. The various models, modules, systems, and components illustrated in FIG. 1 can exist, if at all, across a plurality of servers and devices. For example, some embodiments can include two servers. The two servers can be communicatively coupled using any suitable communications connection (e.g., using a WAN, a LAN, a wired connection, an intranet, or the Internet).

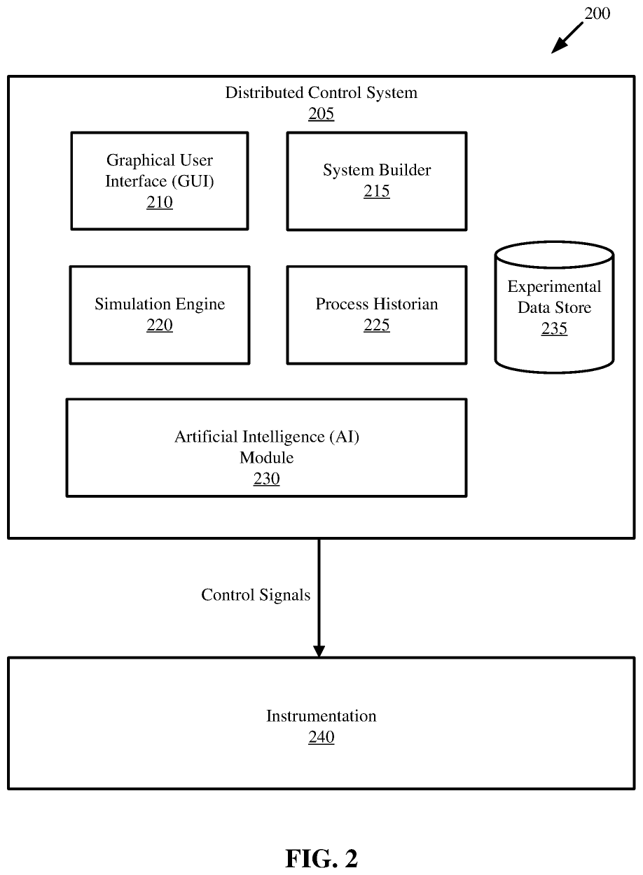

Referring now to FIG. 2, shown is a block diagram of an example computing environment 200 including a distributed control system 205 communicatively coupled to instrumentation 240, in accordance with embodiments of the present disclosure. The distributed control system 205 includes a graphical user interface (GUI) 210, a system builder 215, a simulation engine 220, a process historian 225, and an artificial intelligence (AI) module 230. In embodiments, the functionalities of the system builder 215, simulation engine 220, process historian 225, and AI module 230 can be processor-executable instructions executable by one or more processing circuits based on received inputs.

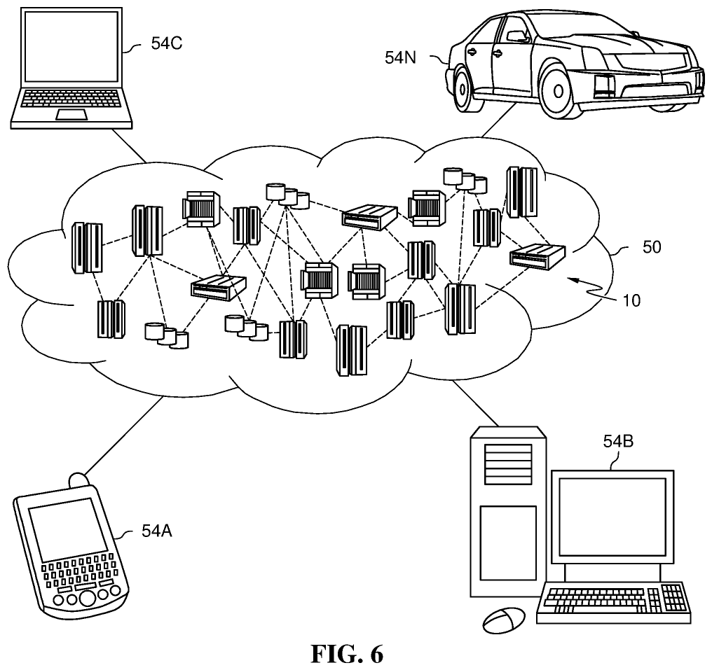

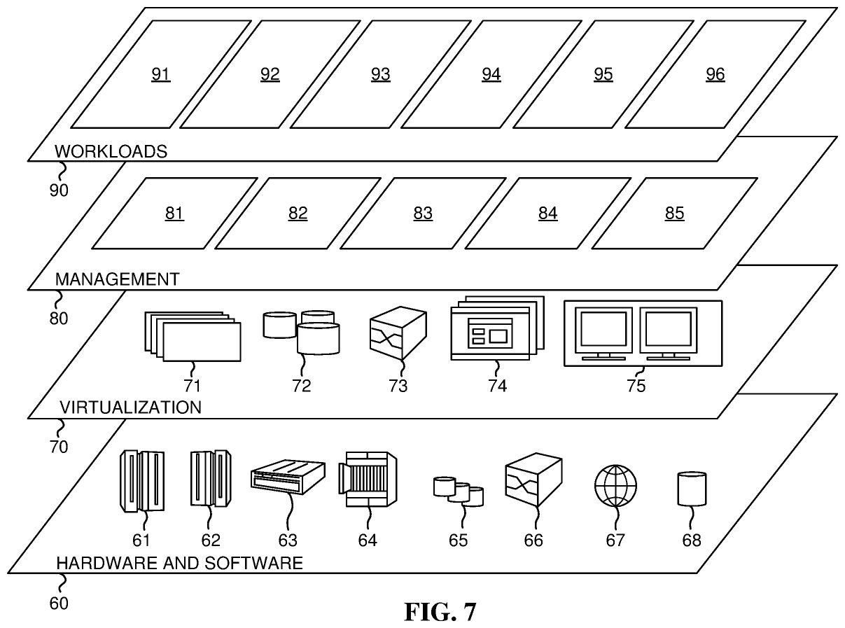

The various models, systems, modules, and components illustrated within the distributed control system 205 can exist, if at all, across a plurality of computing devices. For example, the system builder 215 can be located on a first server, the simulation engine 220 and process historian 225 can be located on a second server, and the AI module 230 can be distributed across various computing devices. Thus, the components described within FIG. 2 can be configured within a cloud computing environment (e.g., the cloud computing environments described throughout FIGS. 6-7). In embodiments, the functionalities of the distributed control system 205 can be the same as, or substantially similar, to the distributed control management application 160 described with respect to FIG. 1.

The graphical user interface (GUI) 210 can facilitate user control of components of the distributed control system 205. In embodiments, the GUI 210 can be accessed locally or remotely. The GUI 210 can facilitate user control of the system builder 215, simulation engine 220, process historian 225, and AI module 230.

The system builder 215 can facilitate process configuration design (e.g., a chemical plant, a chemical process, etc.) using available unit operations. The system builder 215 can provide functionalities to allow a user to define the number of unit operations, type of unit operations (e.g., a batch reactor versus a continuous stirred-tank reactor), settings of unit operations (e.g., temperature and pressure settings), timing of unit operations (e.g., reaction time, heating time, centrifuge time, etc.), reagent input characteristics (e.g., characteristics of one or more chemical compounds input into the process throughout various stages), mass flow, and other variables within the process. Thus, the system builder allows a user to generate process configurations suitable for generating desired products.

The simulation engine 220 can be configured to simulate the process configurations generated by the system builder 215. In particular, the simulation engine 220 can simulate the steps of the process over time based on mathematical models (e.g., reaction kinetics, thermodynamics, fluid dynamics, and mass transport). For example, the simulation engine 220 can simulate unit operations such as separation (e.g., calculated separation based on variables such as speed, time, partition coefficient, and solubility parameters using physical models), heating (e.g., calculated temperature based on variables such as time, power, pressure, mass, and volume using thermodynamic models), and mixing (e.g., calculated chemical compositions based on variables such as mix speed, flow rate, and time using models of fluid dynamics). Further, the chemical composition in each volume unit of the reactor system at any processing time can be determined based on kinetic models. However, the simulation engine 220 can be configured to simulate any suitable unit operation (e.g., homogenization, evaporation, filtration, extraction, etc.) and/or step based on mathematical models. Moreover, the simulation engine 220 can simulate any suitable combination and/or sequence of unit operations to provide an actionable understanding of the overall chemical process.

The characteristics of the reagents within each stage (e.g., within pipes, vessel, and/or instrumentation) and/or each timing (e.g., based on the polling frequency of the simulation) of the process can be recorded by the process historian 225. The process historian 225 can receive the experimental data and store the data within the experimental data store 235. This allows a user to view characteristics of the process (e.g., chemical compounds characteristics, unit operation characteristics, etc.) throughout physical locations and/or timings on the GUI 210. The user can then alter the variables of the process configuration within the system builder 215 based on analysis of the experimental data store.

The AI module 230 can be configured to suggest modifications to (or alternatively, make modifications to) the process configurations built within the system builder 215. In particular, the AI module can analyze data recorded by the process historian 225 such that adjustments can be made to process configurations to improve product yield, improve product quality, reduce cost, reduce environmental impact, reduce process time, reduce physical space occupied, or any other desired goal and/or combination thereof.

In some embodiments, reinforcement learning (RL) can be used to reach desired process configurations based on pre-defined goals (e.g., product identity, product characteristics, product yield, quality, time, cost, etc.). In these embodiments, modifications (e.g., incremental changes) can be made to one or more variables (e.g., chemical composition, type of unit operations, number of unit operations, sequence of unit operations, residence times, etc.) within the process configuration. Simulations can be run for each modified configuration, and reward can be calculated based on each respective configuration's ability to reach the desired goal(s). In these embodiments, a large number (e.g., millions) of simulations can be run until a desired process configuration is identified (e.g., based on an optimization function). This can be completed based on an optimization function that maximizes cumulative reward.

In some embodiments, supervised learning (e.g., active learning) can be used to reach desired process configurations based on pre-defined goals. In these embodiments, user labeling can be received indicating sought values of data points (e.g., chemical concentration, mass, volume, etc.) at particular steps and/or timings within a given process configuration simulation. The process configuration can then be modified based on the labeling, and additional simulations can be run to check whether the modification aided in providing the sought value. This can be completed iteratively until a desired process configuration is identified.

Machine learning algorithms that can be used to improve process configurations include but are not limited to decision tree learning, association rule learning, artificial neural networks, deep learning, inductive logic programming, support vector machines, clustering, Bayesian networks, reinforcement learning, representation learning, similarity/metric training, sparse dictionary learning, genetic algorithms, rule-based learning, and/or other machine learning techniques.

For example, the machine learning algorithms can utilize one or more of the following example techniques: K-nearest neighbor (KNN), learning vector quantization (LVQ), self-organizing map (SOM), logistic regression, ordinary least squares regression (OLSR), linear regression, stepwise regression, multivariate adaptive regression spline (MARS), ridge regression, least absolute shrinkage and selection operator (LASSO), elastic net, least-angle regression (LARS), probabilistic classifier, naïve Bayes classifier, binary classifier, linear classifier, hierarchical classifier, canonical correlation analysis (CCA), factor analysis, independent component analysis (ICA), linear discriminant analysis (LDA), multidimensional scaling (MDS), non-negative metric factorization (NMF), classification and regression tree (CART), chi-squared automatic interaction detection (CHAID), expectation-maximization algorithm, feedforward neural networks, logic learning machine, self-organizing map, single-linkage clustering, fuzzy clustering, hierarchical clustering, Boltzmann machines, convolutional neural networks, recurrent neural networks, hierarchical temporal memory (HTM), and/or other machine learning techniques.

Upon selecting a process configuration, the distributed control system 205 can be configured to issue control signals to instrumentation 240 (e.g., over a network) such that the instrumentation is reconfigured to execute the process. That is, one or more settings of instrumentation used to carry out unit operations can be remotely modified per the selected process configuration such that the process can be executed in the real-world. In some embodiments, user intervention may be required to aid in reaching the selected process configuration.

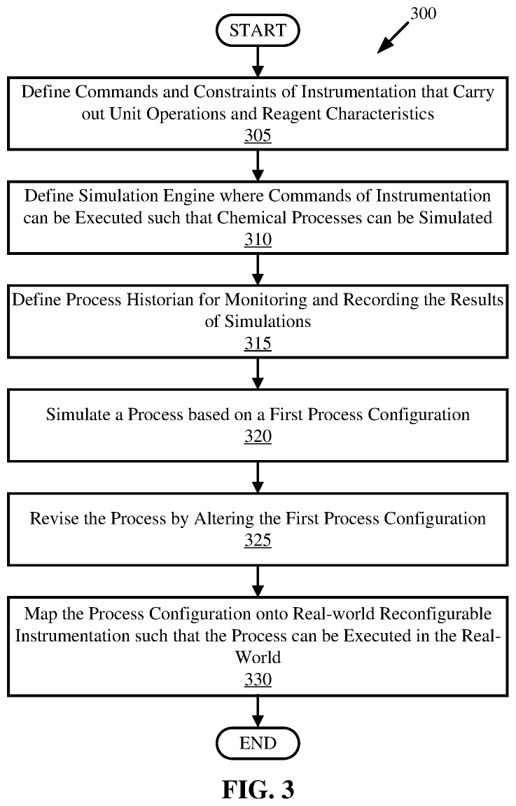

Referring now to FIG. 3, shown is a flow diagram illustrating an example method 300 for configuring and utilizing a distributed control system, in accordance with embodiments of the present disclosure. One or more operations of method 300 can be completed by one or more processing circuits.

Method 300 initiates at operation 305, where commands and constraints of instrumentation that carry out unit operations and reagent characteristics are defined. This can include specifying scripts (e.g., computer instructions of executable commands) for each available item of instrumentation used to carry out unit operations. For example, scripts can define the functions and commands of actuators associated with each item instrumentation. These scripts can be used for process simulation. Further, characteristics of available reagents (e.g., chemical compounds) can also be defined. This can further be used to simulate the process based on mathematical models. In some embodiments, additional information such as cost of resources (e.g., cost of instrumentation and chemicals), physical attributes (e.g., dimensions), and/or environmental impact of aspects can also be defined. This can be used for the purpose of considering cost, physical space occupied, and/or environmental impact during process configuration design.

A simulation engine is then defined where commands of instrumentation that carry out unit operations can be executed such that chemical processes can be simulated. This is illustrated at operation 310. The simulation engine can be built using mathematical models that can imitate how the process occurs in reality. For example, thermodynamic, chemical, and physics models can be used to simulate a process defined by a process configuration.

A process historian for monitoring and recording the results of the simulations is then defined. This is illustrated at operation 315. The process historian can store data regarding the simulated process configuration, the commands issued by instrumentation integrated into the configuration, residence times of reagents, reagent characteristics (e.g., concentration), and product yield over time, among other data.

A process is then simulated based on a first process configuration. This is illustrated at operation 320. As an example, the first process configuration can be defined based on the input reagent characteristics, the number of unit operations, the type of unit operations, the actions taken by the instrumentation, and the timing of unit operations. The first process configuration is then altered. This is illustrated at operation 325. In embodiments, the first process configuration can be manually altered by a user or automatically altered based on AI-embedded capabilities.

The process configuration is then mapped onto real-world reconfigurable instrumentation such that the process can be executed in the real-world. This is illustrated at operation 330. In embodiments, command signals can be transmitted to instrumentation such that the instrumentation carries out the process defined by the process configuration. In some embodiments, user intervention may be required to adjust one or more process configuration variables in the real-world.

The aforementioned operations can be completed in any order and are not limited to those described. Additionally, some, all, or none of the aforementioned operations can be completed while still remaining within the spirit and scope of the present disclosure. For example, in some embodiments, operation 325 may not be completed as the process configuration may not be revised.

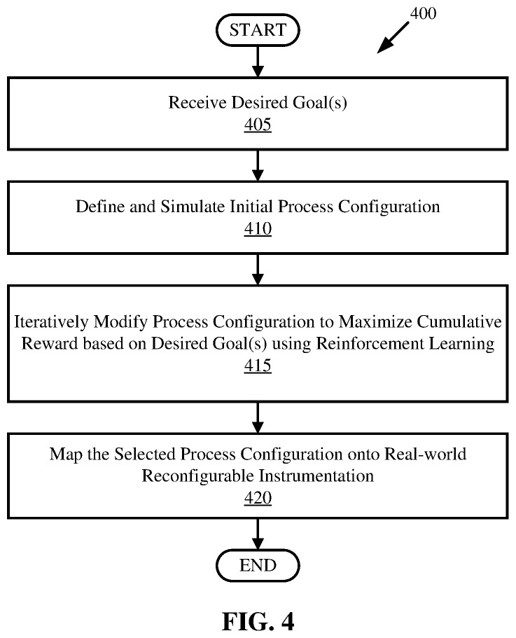

Referring now to FIG. 4, shown is a flow diagram illustrating an example method 400 for modifying an initial process configuration using reinforcement learning, in accordance with embodiments of the present disclosure. One or more operations of method 400 can be completed by one or more processing circuits.

Method 400 initiates at operation 405, where the desired goal(s) are received. Desired goals can be set based on product yield, product quality, cost, environmental impact, process time, and physical space, among others. An initial process configuration can then be defined and simulated. This is illustrated at operation 410. The initial process configuration can be defined to attempt to meet the goals defined at operation 405.

The process configuration is then iteratively modified to maximize cumulative reward based on the set desired goal(s) using reinforcement learning. This is illustrated at operation 415. That is, process configuration variables such as the number of unit operations, type of unit operations, timing of unit operations, order of unit operations, chemical species characteristics (e.g., concentration), mass flow, and others can be altered in an iterative manner (e.g., where one or more of the variables can be changed each iteration) until cumulative reward calculated by the reinforcement learning algorithm is maximized. As discussed herein, “maximization” may refer to a maximum reward over a given number of iterations or over a given processing time. Maximization does not necessarily mean that the process configuration is entirely optimized (or that the cumulative reward cannot increase over a larger time period or more iterations).

The selected process configuration is then mapped onto real-world reconfigurable instrumentation. This is illustrated at operation 420. Thus, the process can be executed by instructing real-world instrumentation to execute the commands defined within the process configuration.

The aforementioned operations can be completed in any order and are not limited to those described. Additionally, some, all, or none of the aforementioned operations can be completed while still remaining within the spirit and scope of the present disclosure.

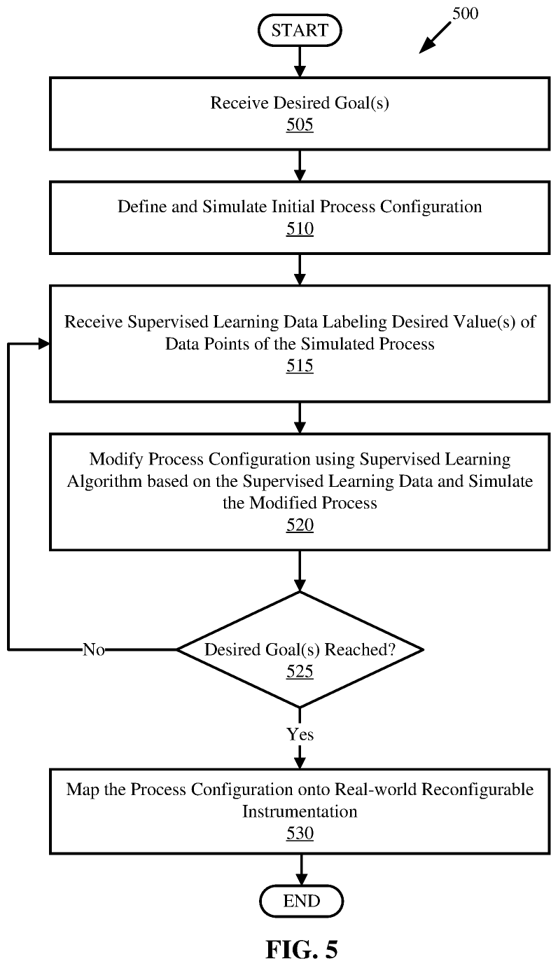

Referring now to FIG. 5, shown is a flow diagram illustrating an example method 500 for modifying an initial process configuration using supervised learning, in accordance with embodiments of the present disclosure. One or more operations of method 500 can be completed by one or more processing circuits.

Method 500 initiates at operation 505, where the desired goal(s) are received. Desired goals can be set based on product yield, product quality, cost, environmental impact, process time, and physical space, among others. An initial process configuration can then be defined and simulated. This is illustrated at operation 510. The initial process configuration can be defined to attempt to meet the goals defined at operation 505.

Supervised learning data labeling one or more desired values of data points of the simulated process is then received. This is illustrated at operation 515. In embodiments, the supervised learning data can be received by request after the initial process configuration is simulated. Values of specific data points at particular time periods can be selected and specified by a user. For example, if a given compound was 40° Celsius at 500 seconds into the process simulation, but the user desires the compound to be 43° Celsius at 500 seconds, the user can transmit supervised learning data to the system indicating that the desired temperature value of the compound should be 40° Celsius at 500 seconds.

The process configuration is then modified using a supervised learning algorithm based on the received supervised learning data and the process is simulated. This is illustrated at operation 520. For example, process configuration variables such as the number of unit operations, type of unit operations, timing of unit operations, order of unit operations, chemical species characteristics (e.g., concentration), and/or mass flow can be modified using a supervised learning algorithm to attempt to reach the specified user value. The modified process configuration is then simulated.

A determination is then made whether the desired goal(s) are reached after the process configuration is modified. This is illustrated at operation 525. This can be completed by analyzing the simulating of the modified process to determine whether the goal(s) specified at operation 505 are met. If the desired goal(s) are not reached, then additional supervised learning data labeling desired value(s) of data points can be received by a user. Thus, method 500 may continuously loop between operations 515 and 525 until the desired goal(s) are reached.

If a determination is made that the desired goal(s) are met, then the selected process configuration is mapped onto real-world reconfigurable instrumentation. This is illustrated at operation 530. Thus, the process can be executed by instructing real-world instrumentation to execute the commands defined within the process configuration.

The aforementioned operations can be completed in any order and are not limited to those described. Additionally, some, all, or none of the aforementioned operations can be completed, while still remaining within the spirit and scope of the present disclosure.

It is to be understood that although this disclosure includes a detailed description of cloud computing, implementation of the teachings recited herein is not limited to a cloud computing environment. Rather, embodiments of the present disclosure are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure, including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure, including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service-oriented with a focus on statelessness, low coupling, modularity, and semantic interoperability. At the heart of cloud computing is an infrastructure that includes a network of interconnected nodes.

Referring now to FIG. 6, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A (e.g., devices 105), desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms, and/or software as services for which a cloud consumer does not need to maintain resources on a local computing device. It is understood that the types of computing devices 54A-N shown in FIG. 6 are intended to be illustrative only and that computing nodes 10 and cloud computing environment 50 can communicate with any type of computerized device over any type of network and/or network addressable connection (e.g., using a web browser).

Referring now to FIG. 7, a set of functional abstraction layers provided by cloud computing environment 50 (FIG. 6) is shown. It should be understood in advance that the components, layers, and functions shown in FIG. 7 are intended to be illustrative only, and embodiments of the disclosure are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and process configuration generation and simulation 96.

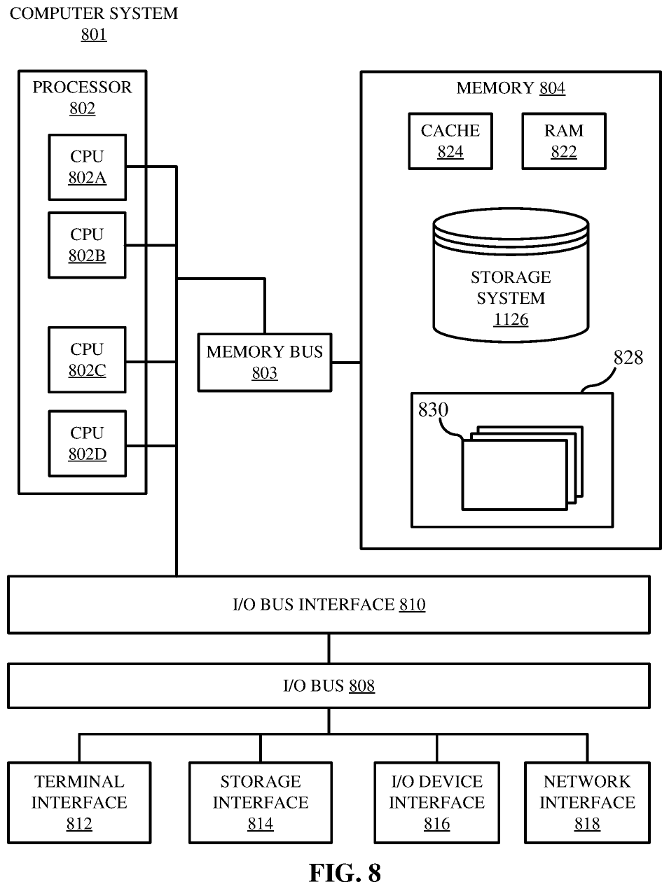

Referring now to FIG. 8, shown is a high-level block diagram of an example computer system 801 (e.g., devices 105, server 135, distributed control system 205) that may be used in implementing one or more of the methods, tools, and modules, and any related functions, described herein (e.g., using one or more processor circuits or computer processors of the computer), in accordance with embodiments of the present disclosure. In some embodiments, the major components of the computer system 801 may comprise one or more CPUs 802, a memory subsystem 804, a terminal interface 812, a storage interface 814, an I/O (Input/Output) device interface 816, and a network interface 818, all of which may be communicatively coupled, directly or indirectly, for inter-component communication via a memory bus 803, an I/O bus 808, and an I/O bus interface unit 810.

The computer system 801 may contain one or more general-purpose programmable central processing units (CPUs) 802A, 802B, 802C, and 802D, herein generically referred to as the CPU 802. In some embodiments, the computer system 801 may contain multiple processors typical of a relatively large system; however, in other embodiments the computer system 801 may alternatively be a single CPU system. Each CPU 802 may execute instructions stored in the memory subsystem 804 and may include one or more levels of on-board cache.

System memory 804 may include computer system readable media in the form of volatile memory, such as random access memory (RAM) 822 or cache memory 824. Computer system 801 may further include other removable/non-removable, volatile/non-volatile computer system storage media. By way of example only, storage system 826 can be provided for reading from and writing to a non-removable, non-volatile magnetic media, such as a “hard-drive.” Although not shown, a magnetic disk drive for reading from and writing to a removable, non-volatile magnetic disk (e.g., a “floppy disk”), or an optical disk drive for reading from or writing to a removable, non-volatile optical disc such as a CD-ROM, DVD-ROM or other optical media can be provided. In addition, memory 804 can include flash memory, e.g., a flash memory stick drive or a flash drive. Memory devices can be connected to memory bus 803 by one or more data media interfaces. The memory 804 may include at least one program product having a set (e.g., at least one) of program modules that are configured to carry out the functions of various embodiments.

One or more programs/utilities 828, each having at least one set of program modules 830 may be stored in memory 804. The programs/utilities 828 may include a hypervisor (also referred to as a virtual machine monitor), one or more operating systems, one or more application programs, other program modules, and program data. Each of the operating systems, one or more application programs, other program modules, and program data or some combination thereof, may include an implementation of a networking environment. Programs 828 and/or program modules 830 generally perform the functions or methodologies of various embodiments.

Although the memory bus 803 is shown in FIG. 8 as a single bus structure providing a direct communication path among the CPUs 802, the memory subsystem 804, and the I/O bus interface 810, the memory bus 803 may, in some embodiments, include multiple different buses or communication paths, which may be arranged in any of various forms, such as point-to-point links in hierarchical, star or web configurations, multiple hierarchical buses, parallel and redundant paths, or any other appropriate type of configuration. Furthermore, while the I/O bus interface 810 and the I/O bus 808 are shown as single respective units, the computer system 801 may, in some embodiments, contain multiple I/O bus interface units 810, multiple I/O buses 808, or both. Further, while multiple I/O interface units are shown, which separate the I/O bus 808 from various communications paths running to the various I/O devices, in other embodiments some or all of the I/O devices may be connected directly to one or more system I/O buses.

In some embodiments, the computer system 801 may be a multi-user mainframe computer system, a single-user system, or a server computer or similar device that has little or no direct user interface, but receives requests from other computer systems (clients). Further, in some embodiments, the computer system 801 may be implemented as a desktop computer, portable computer, laptop or notebook computer, tablet computer, pocket computer, telephone, smart phone, network switches or routers, or any other appropriate type of electronic device.

It is noted that FIG. 8 is intended to depict the representative major components of an exemplary computer system 801. In some embodiments, however, individual components may have greater or lesser complexity than as represented in FIG. 8, components other than or in addition to those shown in FIG. 8 may be present, and the number, type, and configuration of such components may vary.

As discussed in more detail herein, it is contemplated that some or all of the operations of some of the embodiments of methods described herein can be performed in alternative orders or may not be performed at all; furthermore, multiple operations can occur at the same time or as an internal part of a larger process.

The present disclosure can be a system, a method, and/or a computer program product. The computer program product can include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure can be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions can also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams can represent a module, segment, or portion of instructions, which comprises one or more executable instructions for implementing the specified logical function(s). In some alternative implementations, the functions noted in the block can occur out of the order noted in the figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The terminology used herein is for the purpose of describing particular embodiments only and is not intended to be limiting of the various embodiments. As used herein, the singular forms “a,” “an,” and “the” are intended to include the plural forms as well, unless the context clearly indicates otherwise. It will be further understood that the terms “includes” and/or “including,” when used in this specification, specify the presence of the stated features, integers, steps, operations, elements, and/or components, but do not preclude the presence or addition of one or more other features, integers, steps, operations, elements, components, and/or groups thereof. In the previous detailed description of example embodiments of the various embodiments, reference was made to the accompanying drawings (where like numbers represent like elements), which form a part hereof, and in which is shown by way of illustration specific example embodiments in which the various embodiments can be practiced. These embodiments were described in sufficient detail to enable those skilled in the art to practice the embodiments, but other embodiments can be used and logical, mechanical, electrical, and other changes can be made without departing from the scope of the various embodiments. In the previous description, numerous specific details were set forth to provide a thorough understanding the various embodiments. But, the various embodiments can be practiced without these specific details. In other instances, well-known circuits, structures, and techniques have not been shown in detail in order not to obscure embodiments.

Different instances of the word “embodiment” as used within this specification do not necessarily refer to the same embodiment, but they can. Any data and data structures illustrated or described herein are examples only, and in other embodiments, different amounts of data, types of data, fields, numbers and types of fields, field names, numbers and types of rows, records, entries, or organizations of data can be used. In addition, any data can be combined with logic, so that a separate data structure may not be necessary. The previous detailed description is, therefore, not to be taken in a limiting sense.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Although the present disclosure has been described in terms of specific embodiments, it is anticipated that alterations and modification thereof will become apparent to the skilled in the art. Therefore, it is intended that the following claims be interpreted as covering all such alterations and modifications as fall within the true spirit and scope of the disclosure.

Several examples will now be provided to further clarify various aspects of the present disclosure:

Example 1: A method comprising defining commands and constraints of reconfigurable instrumentation that execute unit operations of processes. The method further comprises defining a simulation engine configured to execute commands of the reconfigurable instrumentation such that processes can be simulated based on pre-defined mathematical models. The method further comprises defining a process historian configured to monitor and record results of the simulation. The method further comprises simulating a first process based on a first process configuration. The method further comprises mapping the first process configuration onto real-world reconfigurable instrumentation within the first process configuration.

Example 2: The limitations of Example 1, wherein the method further comprises revising the first process by altering the first process configuration and mapping the revised process configuration onto the real-world reconfigurable instrumentation within the revised process configuration.

Example 3: The limitations of any of Examples 1-2, wherein the first process is revised using reinforcement learning.

Example 4: The limitations of any of Examples 1-3, wherein the first process is revised using supervised learning.

Example 5: The limitations of any of Examples 1-4, wherein the first process configuration is defined by at least a first number of unit operations, a first order of unit operations, a first set of chemical reagents input into the first process.

Example 6: The limitations of any of Examples 1-5, wherein the method further comprises defining at least one goal for the first process, modifying, in an iterative manner, the first process configuration to maximize cumulative reward based on the at least one goal using reinforcement learning, mapping the modified process configuration onto real-world reconfigurable instrumentation within the modified process configuration.

Example 7: The limitations of Example 6, wherein the at least one goal is based on cost and product yield.

Example 8: The limitations of Examples 6-7, wherein the at least one goal is based on environmental impact and product quality.

Example 9: A system comprising one or more processors and one or more computer-readable storage media collectively storing program instructions which, when executed by the one or more processors, are configured to cause the processor to perform a method according to any of Examples 1-8.

Example 10: A computer program product comprising one or more computer readable storage media, and program instructions collectively stored on the one or more computer readable storage media, the program instructions comprising instructions configured to cause one or more processors to perform a method according to any one of Examples 1-8.

What is claimed is:

1. A method comprising:
defining commands and constraints of reconfigurable instrumentation that execute unit operations of processes;
defining a simulation engine configured to execute commands of the reconfigurable instrumentation such that processes can be simulated based on pre-defined mathematical models;
defining a process historian configured to monitor and record results of the simulation;
simulating a first process based on a first process configuration; and
mapping the first process configuration onto real-world reconfigurable instrumentation within the first process configuration.

2. The method of claim 1, further comprising:
revising the first process by altering the first process configuration; and
mapping the revised process configuration onto the real-world reconfigurable instrumentation within the revised process configuration.

3. The method of claim 2, wherein the first process is revised using reinforcement learning.

4. The method of claim 2, wherein the first process is revised using supervised learning.

5. The method of claim 1, wherein the first process configuration is defined by at least a first number of unit operations, a first order of unit operations, and a first set of chemical reagents input into the first process.

6. The method of claim 1, further comprising:
defining at least one goal for the first process;
modifying, in an iterative manner, the first process configuration to maximize cumulative reward based on the at least one goal using reinforcement learning; and
mapping the modified process configuration onto real-world reconfigurable instrumentation within the modified process configuration.

7. A system comprising:
one or more processors; and
one or more computer-readable storage media storing program instructions which, when executed by the one or more processors, are configured to cause the one or more processors to perform a method comprising:
defining commands and constraints of reconfigurable instrumentation that execute unit operations of processes;
defining a simulation engine configured to execute commands of the reconfigurable instrumentation such that processes can be simulated based on pre-defined mathematical models;
defining a process historian configured to monitor and record results of the simulation;
simulating a first process based on a first process configuration; and
mapping the first process configuration onto real-world reconfigurable instrumentation within the first process configuration.

8. The system of claim 7, wherein the method performed by the one or more processors further comprises:
revising the first process by altering the first process configuration; and
mapping the revised process configuration onto the real-world reconfigurable instrumentation within the revised process configuration.

9. The system of claim 8, wherein the first process is revised using reinforcement learning.

10. The system of claim 8, wherein the first process is revised using supervised learning.

11. The system of claim 8, wherein the first process configuration is defined by at least a first number of unit operations, a first order of unit operations, and a first set of chemical reagents input into the first process.

12. The system of claim 7, wherein the method performed by the one or more processors further comprises:
defining at least one goal for the first process;
modifying, in an iterative manner, the first process configuration to maximize cumulative reward based on the at least one goal using reinforcement learning; and
mapping the modified process configuration onto real-world reconfigurable instrumentation within the modified process configuration.

13. The system of claim 12, wherein the at least one goal is based on cost and product yield.

14. The system of claim 12, wherein the at least one goal is based on environmental impact and product quality.

15. A computer program product comprising one or more computer readable storage media, and program instructions collectively stored on the one or more computer readable storage media, the program instructions comprising instructions configured to cause one or more processors to perform a method comprising:
defining commands and constraints of reconfigurable instrumentation that execute unit operations of processes;
defining a simulation engine configured to execute commands of the reconfigurable instrumentation such that processes can be simulated based on pre-defined mathematical models;
defining a process historian configured to monitor and record results of the simulation;
simulating a first process based on a first process configuration; and
mapping the first process configuration onto real-world reconfigurable instrumentation within the first process configuration.

16. The computer program product of claim 15, wherein the method performed by the one or more processors further comprises:
revising the first process by altering the first process configuration; and
mapping the revised process configuration onto the real-world reconfigurable instrumentation within the revised process configuration.

17. The computer program product of claim 16, wherein the first process is revised using reinforcement learning.

18. The computer program product of claim 16, wherein the first process is revised using supervised learning.

19. The computer program product of claim 16, wherein the first process configuration is defined by at least a first number of unit operations, a first order of unit operations, and a first set of chemical reagents input into the first process.

20. The computer program product of claim 15, wherein the method performed by the one or more processors further comprises:
defining at least one goal for the first process;
modifying, in an iterative manner, the first process configuration to maximize cumulative reward based on the at least one goal using reinforcement learning; and
mapping the modified process configuration onto real-world reconfigurable instrumentation within the modified process configuration.
